(19) United States
(12) Patent Application Publication
(10) Pub. No.: US 2020/0390902 A1
(43) Pub. Date: Dec. 17, 2020

(54) Antibody-Drug Conjugates Targeted at Human Aspartyl-(Asparaginyl)-B-Hydroxylase (HAAH)

(71) Applicant: Panacea Pharmaceuticals, Inc., Gaithersburg, MD (US)

(72) Inventors: Hossein Ghanbari, Gaithersburg, MD (US); Steven A. Fuller, Gaithersburg, MD (US); Michael Lebowitz, Pikesville, MD (US); Zhi-Gang Jiang, Gaithersburg, MD (US)

(21) Appl. No.: 16/108,026

(22) Filed: Aug. 21, 2018

Related U.S. Application Data
(60) Provisional application No. 62/548,732, filed on Aug. 22, 2017.

(51) Int. Cl. A61K 47/68; C07K 16/40; A61P 35/00

(52) U.S. Cl. CPC A61K 47/6871; C07K 16/40; A61P 35/00; A61K 47/6889; A61K 47/6803

(57) ABSTRACT

Disclosed are anti-HAAH antibodies and derivatives thereof conjugated to cytotoxic, cytostatic, immunosuppressive, or other therapeutic agents, as well as pharmaceutical compositions comprising the antibody- and antibody derivative-drug conjugates. Also disclosed are methods for the treatment of HAAH-expressing cancers, comprising administering to a subject the disclosed pharmaceutical compositions.

### FIELD OF THE MENTION

The present disclosure relates to means and methods for specific delivery of drugs to diseased cells. More particularly, the present invention relates to antibody-drug conjugates and methods of using the same:

### BACKGROUND OF THE INVENTION

Antibody-drug conjugates (ADCs) comprise an antibody that is specific for a disease antigen, a linker and one or more drug molecules. The goal is to utilize the antibody as a means of specific delivery of the drug to diseased cells. Theoretically this should decrease systemic exposure to the drug and allow for use of a lower dose to avoid the severe side effects of drug therapy. In cancer, the approach employs an antibody-drug conjugate to target a tumor associated antigen and elicit cancer-specific cytotoxicity. To date there have been three ADCs approved by the US FDA for cancer treatment and many more are now in the development stage. The efficacy of any ADC is dependent on three factors; the antibody, the attached drug, and the linker used to connect them. While each of these factors is of import, with the growth of interest in ADCs for cancer therapy, the use of specific drugs and linkers has become more routine. Thus, the specificity of the tumor-associated antigen and the antibody used to target it are now, perhaps, the primary obstacles towards the development of novel ADCs.

Human aspartyl (asparaginyl) β-hydroxylase (HAAH), also known as aspartate β-hydroxylase (ASPH) is an embryonic/developmental protein, which is downregulated in normal cells after birth but overexpressed on the surface of many malignant cells. It has been demonstrated to be sufficient to induce cancer cell proliferation, motility, and invasiveness. The enzyme hydroxylates important residues in the NOTCH protein altering signal transduction pathways that lead to increased growth and metastatic potential. Increased levels of HAAH have been detected by immunohistochemistry in a diverse array of solid and hematological cancers (n>20), including: liver, bile duct, brain, breast; colon, prostate, ovary, pancreas, and lung cancers as well as various leukemias. HAAH is not found in measurable quantities in normal tissue (n>500) including normal adjacent tissue within cancer biopsy specimens or in benign proliferative disorders.

There is a need in the art for developing an approach for depleting, inhibiting and/or killing the growth and spread of HAAH-expressing cells involved in cancers. Accordingly, there is a need for anti-HAAH antibody-drug conjugates that are constructed in such a manner so as to be capable of having a clinically useful cytotoxic, cytostatic, gar immunosuppressive effect on HAAH-expressing cells, particularly without exerting undesirable effects on non-HAAH-expressing cells. Such compounds would be useful therapeutic agents against cancers that express HAAH.

### SUMMARY OF THE INVENTION

The present disclosure relates to antibody-drug conjugates.

The present invention further relates to methods of making antibody-drug conjugates.

One embodiment of the present invention encompasses methods of treating cancer comprising contacting a cancer tumor with an antibody-drug conjugate.

An embodiment of the present invention encompasses a fully human anti-HAAH antibody conjugated to an anti-cancer drug. In some embodiments of the invention the cancer drug is auristatin or an auristatin derivative. In some embodiments of the invention the auristatin derivative is monomethyl auristatin E (MMAE) or monomethyl auristatin F (MMAF). In some embodiments of the invention the cancer drug is a maytansinoid or a derivative thereof. In some embodiments of the invention the maytansinoid is mertansine/emtansine (DM1) or ravtansine/soravtansine (DM4), In some embodiments of the invention the cancer drug is a duocarmycin or a derivative thereof. In some embodiments of the invention the duocarmycin is duocarmycin SA (DUO). In some embodiments of the invention the antibody is separated from the cancer drug by at least one linker. In some embodiments of the invention the linker is selected from a non-cleavable linker, a cleavable linker, and a combination thereof. In some embodiments of the invention, the non-cleavable linker is selected from maleimidocaproyl (MC). In some embodiments of the invention, the cleavable linker is selected from valine citruline (vc) combined with para-amino benzyl alcohol (PABA). In some embodiments of the invention, the fully human anti-ASPH antibody has a light chain comprising three complementarity determining regions CDR1, CDR2, and CDR3, where the amino acid sequence of CDR1, CDR2, and CDR3 have the amino acid sequence set forth in SEQ ID NO: 3 SEQ ID NO: 4; and SEQ ID NO: 5, In some embodiments of the invention, the anti-ASPH antibody has a light chain amino acid sequence as set forth in SEQ H) NO: 2, and a heavy chain amino acid sequence set forth in SEQ ID NO: 1. In some embodiments of the invention, SNS622 is the fully human anti-HAAH antibody.

The present invention farther provides pharmaceutical compositions comprising antibody-drug conjugates of the invention and a pharmaceutically acceptable medium.

Also provided herein are treatment methods comprising the administration of compositions comprising the antibody-drug conjugates of the invention in a pharmaceutically acceptable medium to a patient in need of treatment.

Embodiments of the present invention encompass antibody-drug conjugates and derivatives and methods relating to the use of such conjugates to treat HAAH-expressing cancers. The antibody, or other targeting moiety in the antibody-drug conjugate, binds to HAAH. A drug conjugated to the antibody or targeting moiety exerts a cytotoxic or cytostatic effect on HAAH-expressing cells to treat or prevent recurrence of HAAH-expressing cancers.

One embodiment of the present invention encompasses a hilly human anti-HAAH antibody conjugated to anti-cancer drug maytansinoid (DM1).

An embodiment of the present invention encompasses a fully human anti-HAAH antibody conjugated to monomethyl auristatin E (MMAE).

An embodiment of the present invention encompasses a fully human anti-HAAH antibody conjugated to duocarmycin (DUO).

An embodiment of the present invention encompasses a method of manufacturing an ASPH-directed ADC in which using a thiol directed linker-drug conjugate to yield a DAR of 2-8.

### BRIEF DESCRIPTION OF THE FIGURES

FIGS. 1A and 1B depict exemplary ADC constructs comprising a monoclonal antibody conjugated to mertansine (DM1). In FIG. 1A the antibody and drug are connected by the non-cleavable linker maleimidocaproyl (MC), In this figure, the chemical structure for MC is encircled. In FIG. 1B the antibody and drug are connected by a combination of the non-cleavable linker MC, and a combination of the cleavable linkers valine citruline and para-amino benzyl alcohol (vc-PAB). In this figure, the chemical structure for MC is encircled, and the chemical structure for vc-PAB is framed by straight lines.

FIGS. 2A and 2B depict exemplary ADC constructs comprising a monoclonal antibody conjugated to monomethyl auristatin E (MMAE). In FIG. 2A the antibody is connected to the drug by the non-cleavable linker MC. In this figure, the chemical structure for MC is encircled. In FIG. 2B the antibody and drug are connected by a combination of the non-cleavable linker MC and the cleavable linker combination vc-PAB. Framed by straight lines in this figure are the chemical structure for vc-PAB and the protease cleavage site.

FIG. 3 depicts an exemplary ADC construct comprising a monoclonal antibody conjugated to duocarmycin (DUO), connected by a combined linker of MC, a 4-unit polyethylene glycol (PEG4), vc-PAB, and N,N-Dimethylethanolamine (DMEA), In this figure, the chemical structure for vc-PAB is framed by straight lines.

FIGS. 4A to 4D depict hydrophobic interaction chromatographs (HIC) obtained for the drug to antibody ratio (DAR) determination of the different SNS622 ADCs. FIG. 4A: chromatograph obtained from the purified SNS622. FIG. 4B: chromatograph obtained from SNS622-MC-DM1. FIG. 4C: chromatograph obtained from SNS622-MC-vc-PAB-MMAE with a 10:1 DAR for reaction. FIG. 4D: chromatograph obtained from SNS622-MC-PEG4-vc-PAB-DMEA-DUO with a 2.2:1 DAR for reaction. The approximate DAR distribution, or given ratio of payload to antibody is indicated on each peak.

FIGS. 5A and 5B depict graphs of the effects of SNS622 ADCs on cell viability of solid human lung carcinoma cell line H460 and acute myeloid leukemia cell line MOLM-14. FIG. 5A depicts a representative graph of MTS assay results for H460 cells treated with a non-specific human monoclonal control IgG1 (♦); non-conjugated anti-HAAH SNS622 (▪); SNS622-MC-DM1 with a 3.47:1 (10:1 for reaction) DAR (▴); SNS622-MC-vc-PAB-MMAE with a 6.31:1 (10:1 for reaction) DAR (●) and SNS622-MC-PEG4-vc-PAB-DMEA-DUO with a 1.92:1 (2.2:1 for reaction) DAR (X). FIG. 5B depicts a graph of the MTS assay results for MOLM-14 cells treated with non-conjugated SNS622 (▪); SNS622-MC-DM1 with a 3.47:1 (10:1 for reaction) DAR (*), SNS622-MC-vc-PAB MMAE with a 6.31:1 (101 for reaction) DAR (●); and SNS622-MC-PEG4-vc-PAB-DMEA-DUO with a 1.92:1 (2.2:1 for reaction) DAR (▴). The absorbance at 490 nm is shown on the Y axis. The Log of the antibody concentration in nM is shown on the X axis.

FIGS. 6A and 6B depict graphs of the effects of linker and payload on cytotoxicity to cancer cell line H460. FIG. 6A: graph of the H460 cell viability quantified using an MTS assay, and expressed as percentage of untreated control on the Y axis. FIG. 6B: graph of the H460 cell death quantified using lactate dehydrogenase (LDH) release assays, expressed as percentage of 512 nM DM1-elicited maximal LDH release on the Y axis, SNS-622-MC-DM1 (▴) SNS622-MC-vc-PAB-DM1 (♦); SNS622-MC-vc-PAB-MMAE (●); and SNS622-MC-MMAE (▪). The Log of the antibody concentration in nM is shown on the X axis.

FIG. 7 depicts a graph of the effects of DAR and pH on the cytotoxicity of SNS622-DM1 conjugates to cancer cell line H460. The effects on H460 cell viability were determined using an MTS assay alter a 48-hour treatment, Non-conjugated SNS622 (●); DAR 2.5 at pH 7 (▪); DAR 2.5 at pH 8 (□); DAR 5 at pH 7 (♦); DAR 5 at pH 8 (⋄); DAR 10 at pH 7 (▴); DAR 10 at pH 8 (Δ). The absorbance at 490 nm is shown on the Y axis. The Log of the antibody concentration in nM is shown on the X axis.

FIGS. 8A and 8B depict graphs of the effects of DAR, linker, and payload on the cytotoxicity of SNS622-DM1 and SNS622-MMAE to cancer cell line H460. FIG. 8A: graph of the H460 cell viability quantified using an MTS assay, expressed as percentage of untreated control on the Y axis. FIG. 8B: graph of the H460 cell death quantified using lactate dehydrogenase (LDH) release assays, expressed as percentage of 512 nM DM1-elicited maximal LDH release on the Y axis. SNS622-MC-vc-PAB-DM1 with 2.5:1 DAR(▴); SNS622-MC-vc-PAB-DM1 with 5:1 DAR (●); SNS622-MC-vc-PAB-DM1 with 10:1 DAR (▪); SNS622-MC-MMAE with 2.5:1DAR (Δ); SNS622-MC-MMAE with 5:1 DAR (∘); SNS622-MC-MMAE with 10:1 DAR (□).

FIGS. 9A to 9D show phase contrast microscope images of human lung carcinoma cell line H460 cells and normal human lung fibroblast (NHLF) cells either untreated or treated with SNS622-MC-DM1. FIG. 9A: untreated NHLF cells; FIG. 9B: NHLF cells treated with SNS622-MC-DM1; FIG. 9C: untreated H460 cells; FIG. 9D: H460 cells treated with SNS622-MC-DM1.

FIG. 10 depicts a bar graph of the percentage of LDH released by cells treated with SNS622-MC-DM1. In the order shown, bars are: untreated NHLF cells; untreated H460 cells; NHLF cells treated with SNS622-MC-DM1; H460 cells treated with SNS622-MC-DM1. Results are expressed as percent of untreated culture. Means Standard Deviation, N=6.

FIG. 11 depicts a graph of the survival of high ASPH-expressing human pancreatic ductular adenocarcinoma cell line MIA PaCa-2 ASPH treated with SNS622-MC-DM1 (♦) or with IgG1-MC-DM1 control ADC (X) for 48 hours. The relative absorbance is shown on the Y axis. The ADC concentration (in nM) is shown on the X axis. The relative absorbance is expressed as Means±Standard Deviation.

FIG. 12 shows an image of a western blot detecting ASPH and glyceraldehyde-3-phosphate dehydrogenase (GAPDH) in (a) MIA PaCa-2 EV cells and (b) MIA PaCa-2 ASPH cells.

FIG. 13 depicts a diagram of the schedule of treatment with vehicle, IgG1-MC-DM1, or SNS622-MC-DM1 of a mouse model inoculated with pancreas cancer cells,

FIG. 14 shows images of tumors primarily inoculated with MIA PaCa-2 EV and MIA PaCa-2 ASPH after 29 days of treatment with SNS622-MC-DM1.

FIGS. 15A and 15B depict graphs of the tumor volumes of primarily inoculated tumors after treatment for 1, 8, 15, 22, 26, and 29 days. FIG. 15A: volumes (in mm³) of tumors primarily inoculated with MIA PaCa-2 EV (EV) and MIA PaCa-2 ASPH (ASPH) following treatment with 5 mg/kg SNS622-MC-DM1. FIG. 15B: volumes (in mm³) of tumors primarily inoculated with MIA PaCa-2 ASPH and treated with 5 mg/kg vehicle control (C); IgG1-MC-DM (IgG); or SNS622-MC-DM1 (622). Values represent mean±standard error of the mean (S.E.M.) derived from primary tumor volumes per group (n=7 for each group). Statistical analyses between two groups were performed using the Mann-Whitney U test. Symbols to show significant levels in statistical difference are included in the figures.

FIG. 16 depicts a graph of the primary tumor volume in 5 to 6 week-old female Nod Scid Gamma (NSG) mice xenografted (PDx) with human pancreatic ductular adenocarcinoma (PDAC). Experimental control, untreated mice with PDAC xenograft (●); mice treated once per week with 2.5 mg/kg SNS622 antibody (▪); mice treated once per week with 2.5 mg/kg SNS622-MC-DM1 (▴). Tumor size was measured twice a week. Values represent mean±S.E.M, derived from each group (SNS622, n=8; SNS622-MC-DM1 n=8; non-treatment or control, n=5). *P<0.05, **P<0.01, ***P<0.005 versus SNS622 or non-treated control groups.

FIG. 17 depicts macroscopic images of lungs in the PDx model mice untreated (control); treated with SNS622 (622); or treated with 1.9 mg/kg SNS622-MC-DM1 (622-DM1).

FIGS. 18A and 18B depict graphs of the number of metastatic nodules in the same groups as in FIG. 17. FIG. 18A: total number of metastatic nodules in each group quantified after fixation in Bouin's solution. FIG. 18B: mean number of metastatic nodules in each group. Untreated (control); treated with SNS622 (622); treated with 1.9 mg/kg SNS622-MC-DM1 (622-DM1). Values represent mean±S.E.M. derived from pulmonary spread per group (non-treatment control, n=7; 622, n=8; 622-DM1, n=8). Statistical analyses between two groups were performed by Mann-Whitney U test, *P<P=0.024.

### DETAILED DESCRIPTION OF THE INVENTION

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present invention, methods and materials used are now described. All publications mentioned herein are incorporated herein by reference in their entirety.

In the present disclosure, “HAAH” and “ASPH” are used interchangeably and stand for human aspartyl (asparaginyl) beta-hydroxylase, ASPH is expressed by the majority of malignant neoplasms, such as cancers from the liver, bile duct, brain, breast, colon prostate, ovary, pancreas, and lung, and various leukemias.

In the present disclosure, “PAN-622” and “SNS622” are used interchangeably and stand for a high-affinity, fully-humanized monoclonal antibody against human aspartyl (asparaginyl) beta-hydroxylase. The PAN-622 antibody was initially developed and characterized by Yeung Y. A., et al. (2007, “Isolation and Characterization of Human Antibodies Targeting Human Aspartyl (asparaginyl) beta-Hydroxylose,” Hum. Antibodies 16: 163). The amino acid sequences of the light chain, heavy chain, and light chain complementarity determining regions for the PAN-622 antibody are presented in Table I, below:

TABLE 1
PAN-622 Amino Acid Sequences

| | | SEQ |
|---|---|---|
| | SEQUENCE | ID NO: |
| Heavy | QVQLQQSGPGLVKPSQTLSLTCAISGDSVSSNSAAW | SEQ ID |
| Chain | NWIRQSPSRGLEWLGRTYYRSKWYNDYAVSVKSRI | NO: 1 |
| | TINPDTSKNQFSLQLNSVTPEDTAVYYCARTGYSSS | |
| | WVVNFDYWGQGTLVTVSSASTKGPSVFPLAPSSKS | |
| | TSGGTAALGCLVKDYFPEPVTVSWNSGALTSGVHT | |
| | FPAVLQSSGLYSLSSVVTVPSSSLGTQTYICNVNHKP | |
| | SNTKVDKRVEPKSCDKTHTCPPCPAPELLGGPSVFL | |
| | FPPKPKDTLMISRTPEVTCVVVDVSHEDPEVKFNWY | |
| | VDGVEVHNAKTKPREEQYNSTYRVVSVLTVLHQD | |
| | WLNGKEYKCKVSNKALPAPIEKTISKAKGQPREPQV | |
| | YTLPPSREEMTKNQVSLTCLVKGFYPSDIAVEWESN | |
| | GQPENNYKTTPPVLDSDGSFFLYSKLTVDKSRWQQ | |
| | GNVFSCSVMHEALHNHYTQKSLSLSPGK | |
| Light | QPVLTQSPSASGTPGQRVTISCSGSSSNIGSNYVYWY | SEQ ID |
| Chain | QQLPGTAPKLLIYKNNQRPSGVPDRFSGSKSGTAAS | NO: 2 |
| | LAISGLQSEDEADYYCAAWDDSLRGYVFGTGTKLT | |
| | VLGQPKAAPSVTLFPPSSEELQANKATLVCLISDFYP | |
| | GAVTVAWKADSSPVKAGVETTTPSKQSNNKYAASS | |
| | YLSLTPEQWKSHRSYSCQVTHEGSTVEKTVAPTECS | |
| LCDR1 | SGSSSNIGSNYVYWY | SEQ ID |
| | | NO: 3 |
| LCDR2 | KLLIYKNNQRPS | SEQ ID |
| | | NO: 4 |
| LCDR3 | AAWDDSLRGYV | SEQ ID |
| | | NO: 5 |

In the present disclosure, the preparation and testing of ADCs comprising SNS622 are disclosed. These ADCs were prepared by conjugating the high-affinity, fully humanized anti-HAAH antibody SNS622 to mertansine (DM1), monomethyl auristatin E (MMAE), or duocarmycine (DUO), using, as linkers maleimidocaproyl (MC), valine-citrulline (vc), para-amino benzyl alcohol (PAB), a 4-unit polyethylene glycol (PEG4), or a combination of at least two of these linkers

In the present disclosure, “MIA-PaCa-2 HAAH” cells and “MIA-PaCa-2 ASPH” cells are used interchangeably and refer to MIA-PaCa-2 cells that have been transformed to overexpress ASPH.

In the present disclosure “MIA-PaCa-2 EV” cells are MIA-PaCa-2 cells transduced with an empty lentiviral vector.

For simplicity and illustrative purposes, the principles of the present invention are described by referring to various exemplary embodiments thereof. Although the preferred embodiments of the invention are particularly disclosed herein, one of ordinary skill in the art will readily recognize that the same principles are equally applicable to, and can be implemented in other systems, and that any such variation would be within such modifications that do not part from the scope of the present invention. Before explaining the disclosed embodiments of the present invention in detail, it is to be understood that the invention is not limited in its application to the details of any particular arrangement shown, since the invention is capable of other embodiments. The terminology used herein is for the purpose of description and not of limitation. Further, although certain methods are described with reference to certain steps that are presented herein in certain order, in many instances, these steps may be performed in any order as would be appreciated by one skilled in the art, and the methods are not limited to the particular arrangement of steps disclosed herein.

The present disclosure provides antibody-drug conjugates and derivatives and methods relating to the use of such conjugates to treat HAAH-expressing cancers. The antibody, or other targeting moiety in the antibody-drug conjugate, binds to HAIR A drug conjugated to the antibody or targeting moiety exerts a cytotoxic or cytostatic effect on HAAH-expressing cells to treat or prevent recurrence of HAAH-expressing cancers.

A suitable cytotoxic or cytostatic agent useful in the preparation of an ADC can be any agent approved by a healthcare regulatory agency such as the Food and Drug Administration (TDA) in the U.S.A Medicines & healthcare Products Regulatory Agency (MHRA) in the United Kingdom, Therapeutic Goods Administration in Australia, Health Canada in Canada, or European Medicines Agency (EMA) in Europe.

Suitable cytotoxic agents can be, for example, an auristatin, a DNA minor groove binding agent, a DNA minor groove alkylating agent, an enediyne, a lexitropsin, a duocarmycin, taxane, a puromycin, a dolastatin, a maytansinoid, and a vinca alkaloid. In specific embodiments, the drug is cytotoxic agent is APP, MMAF, MMAE, AEB, AEVB, auristatin E, paclitaxel, docetaxel, CC-1065, SN-38, topotecan, morpholino-doxorubicin, rhizoxin, cyanomorpholino-doxambicin, dolastatin-10, echinomycin, combretatstatin, chalicheamicin, maytansine, DM1, or netropsin. Other suitable cytotoxic agents include anti-tubulin agents, such as an auristatin, a vinca alkaloid, a podophyllotoxin, a taxane, a baccatin derivative, a cryptophysin, a maytansinoid, a combretastatin, or a dolastatin. In specific embodiments, the antitubulin agent is AFP, MMAF, MMAE, AEB, AEVB, auristatin E, vincristine, vinblastine, vindesine, vinorelbine, VP-16, camptothecin, paclitaxel, docetaxel, epothilone epothilone B, nocodazole, colchicines, colcimid, estramustine, cemadotin, discodermolide, maytansine, DM-1, or eleutherobin.

Other agents include, for example, ganciclovir, etanercept, cyclosporine, tacrolimus, rapamycin, cyclophosphamide, azathioprine, mycophenolate mofetil, methotrexate, cortisol, aldosterone, dexamethasone, a cyclooxygenase inhibitor, a 5-lipoxygenase inhibitor, or a leukotriene receptor antagonist.

Linkers accommodate different conjugation chemistries on both, antibodies and cytotoxic agents. Linkers contribute to the stability of the ADC complex in systematic circulation and dictate the cytotoxic agent release mechanisms once internalized and trafficked into designated cellular locations. Linkers are commonly categorized as cleavable linkers or non-cleavable linkers, based on their release mechanisms. Examples of cleavable linkers are peptide linkers, β-glucuronide linkers, pH-sensitive linkers, and Glutathione-sensitivity linkers.

Linkers should possess two crucial characteristics. First, linkers should possess stability in plasma for an extended period of time. In this manner the ADCs can reach and localize to the cancer cell in the original formation. After internalization, the linkers play a role as a trigger for releasing the cytotoxic drugs when the ADCs face particular circumstances in the cancer cells and the released cytotoxic drugs then bind to their targets. With the particular microenvironment of tumor cells and the delivery mechanism selected, the active formation of the cytotoxic drug may be efficiently released from ADCs by fracturing the designed linkers inside the target cells. The stability and rupturing capacity of linkers affect the overall pharmacokinetics (PK) properties, toxicities and therapeutic indexes of ADCs.

Linkers are classified in different categories according to the mechanism of drug release and their stability in circulation. Cleavable linkers rely on the physiological environment, such as there being high glutathione concentrations, low pH, and special protease, which could assist the linkers in enabling chemical or biochemical reactions by way of hydrolyzation or proteolysis. Non-cleavable linkers depend on the degradation of the monoclonal antibody after internalization of the ADC within the lysosomes and endosomes to generate the metabolites containing the active cytotoxic drugs with or without a portion of the linkers.

Each release strategy must account for many factors: the various activities of cytotoxic drugs, the characteristics of monoclonal antibodies, the nature and intracellular processing of the target antigen, and the particular disease, Optimal linkers designed to conjugate the cytotoxic drugs to monoclonal antibodies must meet the particular requirements.

Many non-cleavable linkers have been explored in the development of ADCs. The greatest advantage of non-cleavable linkers is their increased plasma stability. Examples of non-cleavable linkers are N-succinimidyl-4-(N-maleimidomethyl) cyclohexane-1-carboxylate (SMCC) linkers, and maleimidocaproyl linkers.

Cleavable linkers are stable in the blood circulation for a long period of time, and are efficiently released in the tumor microenvironment. Chemically labile linkers include acid-cleavable linkers and reducible linkers. Hydrazone and disulfide linkers are examples of acid-cleavable linkers. Enzyme cleavable linkers take advantage of the abundance of hydrolytic enzymes with the specificity to recognize the sequences of peptides or patterns of carbohydrate in order to degrade peptides and carbohydrates.

Peptide-based linkers are designed to keep ADCs intact in systemic circulation, and allow easy release of the cytotoxic drugs upon cleavage by specific intracellular proteases, such as cathepsin B. Examples of peptide-based linkers are valine-citrulline (Val-Cit) dipeptide phenylalanine-lysine (Phe-Lys) dipeptide linker, and β-glucuronide linker.

We have developed a fully human antibody against HAAH, PAN-622, which displays exquisite specificity for cancer. Here we explore PAN-622 drug conjugates for ultimate use in treating both hematological and solid tumors. PAN-622 was conjugated to three different drugs: a maytansinoid (DM1), monomethyl auristatin E (MMAE), or duocarmycin (DUO). DM1 was conjugated via a non-cleavable thioether linker, while MMAE and DUO were conjugated using valine-citruline containing linkers that are cleavable by cathepsin B in the endosomal compartment. Conjugation of drugs to the PAN-622 antibody had little effect on the affinity of the antibody for HAAH. Binding affinities were determined by immunoassay on fixed cancer cells (H460, human lung cancer line) and were ˜0.1, 0.2 and 0.5 nM for PAN-622-DM1, PAN-622-MMAE and PAN-622-DUO, respectively. The affinity of PAN-622 for HAAH as expressed on live cells has previously been shown to be ˜1 nM. Efficacies of the three ADCs for killing of H460 cells were determined using an MTS assay. The PAN-622-DM1 had an EC50 of ˜15 nM. The EC50 for PAN-622-MMAE was ˜60 nM and that for PAN-622-DUO was ˜300 nM, Importantly, both unconjugated PAN-622 and a non-relevant antibody conjugated to MMAE did not display any killing of the H460 cell line. Efficacy was also measured on a representative hematological cancer line, MOLM-14 (acute myelogenous leukemia) where EC50s were in the 20-50 nM range for all three ADCs. This work serves as a proof-of-concept; laying the groundwork for further development of HAAH-targeted ADCs.

The hypotriploid human cell line MIA PaCa-2 was obtained from the American Type Culture Collection (ATTC, Manassas, Va., U.S.A.; catalog No, CRL-1420). This cell line was established in 1975 by Yunis A A, et al. from tumor tissue of the pancreas obtained from a 65-year-old Caucasian male (1977, “Human pancreatic carcinoma (MIA PaCa-2) in continuous culture: sensitivity to asparaginase,” Int. J. Cancer 19: 128).

In the present disclosure, DAGIC1194-DM1 is a control, non-specific monoclonal ADC, The DAGIC1194 antibody was obtained from Creative DIagnostis (Shirley, N.Y., U.S.A.) and labeled by Goodwin Biotechnology, Inc, (Plantation, Fla., U.S.A) using the same procedures as used to label SNS622 with DM1.

### EXAMPLES

### Example 1

### Preparation of Anti-ASPH-Drug Conjugates

Anti-ASPH SNS622 antibody was conjugated to linkers and cancer drugs at Goodwin Biotechnology (Plantation, Fla., U.S.A). Three different cytotoxic drugs were used, mertansine (DM1), monomethyl auristatin F (MMAE), or duocarmycine (DUO).

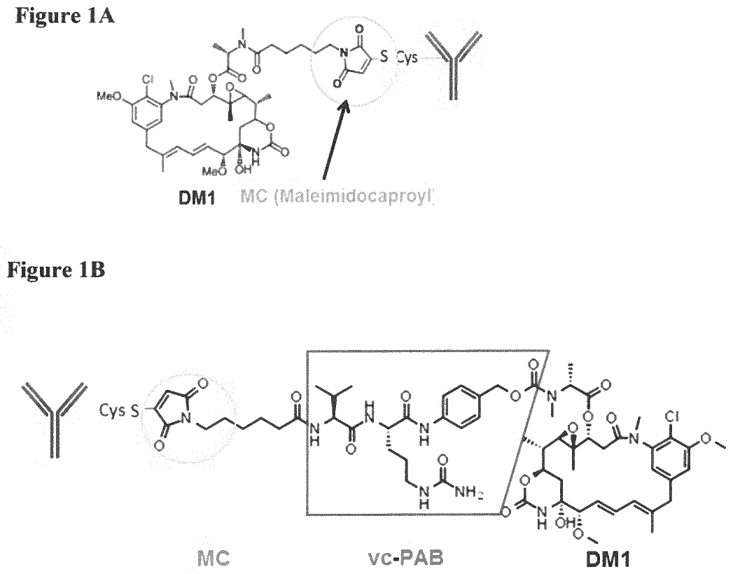

The SNS622 monoclonal antibody was conjugated to DM1, connected by the non-cleavable linker maleimidocaproyl (MC) to prepare SNS622-MC-DM1, or connected by MC and a combination of the cleavable linkers valine citruline and para-amino benzyl alcohol (vc-PAB) to prepare SNS622-MC-vc-PAB-DM1. FIG. 1A depicts an exemplary ADC construct comprising a monoclonal antibody conjugated to DM1 connected by the non-cleavable linker MC. Here, the cytotoxin is released after destruction of the antibody by a protease. FIG. 1B depicts an exemplary ADC construct comprising a monoclonal antibody conjugated to DM1 connected by the non-cleavable linker MC and a combination of the cleavable linkers valine citruline and para amino benzyl alcohol (vc-PAB). In this construct, the cytotoxin is released after Cathespin B protease cleavage.

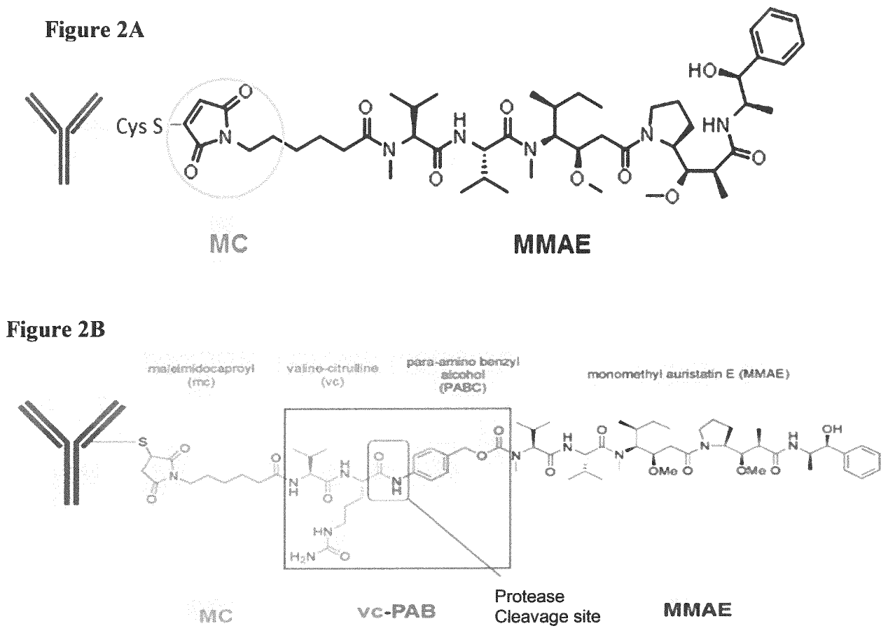

Similarly, the SNS622 monoclonal antibody was conjugated to MMAE connected by MC to prepare SNS622-MC-MMAE or connected by MC and vc-PAB to prepare SNS622-MC-vc-PAB-MMAE. FIG. 2A depicts an exemplary ADC construct comprising a monoclonal antibody conjugated to MMAE connected by the non-cleavable linker MC. The cytotoxin is released after destruction of the antibody by a protease. FIG. 2B depicts an exemplary ADC construct comprising a monoclonal antibody conjugated to MMAE connected by the non-cleavable linker MC and a combination of the cleavable linkers valine citruline and para-amino benzyl alcohol (vc-PAB). In this construct, the cytotoxin is released after Cathespin B protease cleavage.

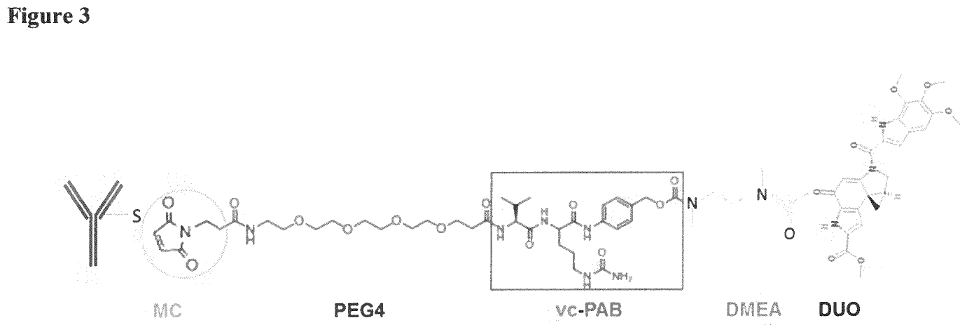

The SNS622 monoclonal antibody was conjugated to duocarmycin (DUO) connected by a combined linker of MC, a four unit of polyethylene glycol (PEG14), vc-PAB, and N,N-Dimethylethanolamine (DMEA) to prepare SNS622-MC-PEG4-vc-PAB-DMEA-DUO. An exemplary ADC construct comprising a monoclonal antibody conjugated to DUO, connected by the linkers MC, PEG4, vc-PAB, and DMEA is depicted in FIG. 3.

Manufacture of ADCs was achieved through thiol-directed chemistry. Briefly linker-drug conjugates were purchased from Levena Biopharma (San Diego, Calif., U.S.A.). Antibody disulfide bonds were reduced by incubation for 1 hour at 25° C. in the presence of Tris(2-carboxyethyl) phosphine (TCEP) at a 5-fold molar excess for DM1 and MMAE conjugations, and at a 1.1 molar excess for DUO conjugation. Subsequently, linker-drug conjugates were added to the reduced antibodies at the indicated challenge ratio and incubated for 1 hour at 25° C. Finally, a molar excess of cysteine was added to quench the unreacted linker-drug moiety. Following the conjugation reaction, all conjugates were purified overnight using a SLIDE-A-LYZER 30 kDa MWCO Dialysis Device (ThermoFisher Scientific) with a total of three (3) buffer exchanges (1×PBS, 7.4) of at least 100 diavolume per dialysis.

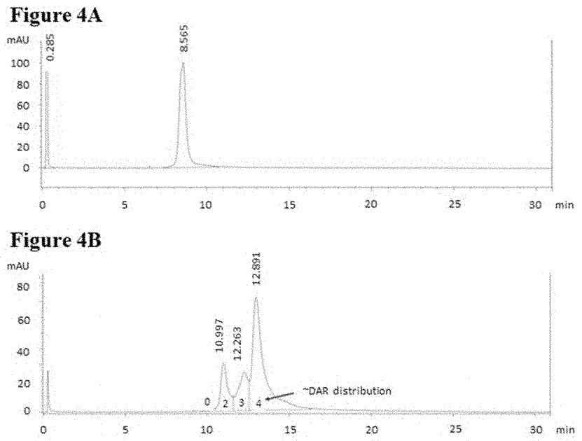

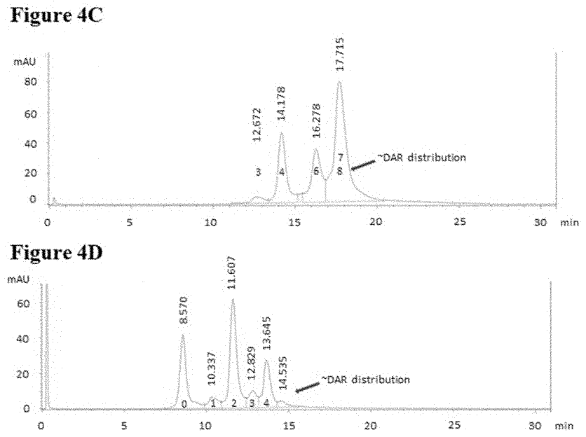

Final Drug Antibody Ratios (DARs) were determined using UV absorption spectroscopy and hydrophobic interaction chemistry (HIC). Chromatograms of the final products are shown in FIGS. 4A to 4D. FIG. 4A shows a chromatogram obtained for non-conjugated SNS622. The monoclonal antibody alone, SNS622 without payload conjugation, peaked at 8.6 minutes. As expected, all SNS622 ADCs showed peaks with an increase in retention time that increased with the higher DARs. In the chromatograms shown in FIGS. 4B to 4D, small numbers under the peaks indicate the approximate DAR for the product:

FIG. 4B shows a chromatogram obtained for SNS622-MC-DM1 prepared with a 10:1 reaction DAR; FIG. 4C shows a chromatogram obtained for SNS622-MC-vc-PAB-MMAE: prepared with a 10:1 reaction DAR; FIG. 4D shows a chromatogram obtained for SNS622-MC-PEG4-vc-PAB-DMEA-DUO obtained with a 2.2:1 reaction DAR. The (A). In ADC products (B-E), all SNS622 antibodies were conjugated with a payload, demonstrated by increases in retention time: The higher DAR, the longer retention time. The approximate DAR in each peak is indicated by a small number.

DAR values derived from HIC were lower than those derived from UV-absorbance, Highly payload-substituted species were less well-resolved on the HIC chromatograms due to peak broadening. DAR values are reported in Table II below.

TABLE II
Final DARs and Bio-efficacies for SNS622
ADCs with Initial Reaction DARs of 10:1

| | | | IC₅₀ | LC₅₀ |
|---|---|---|---|---|
| | Reaction | Final | of MTS | of LDH |
| Name | BAR | DAR | (nM) | (nM) |
| SNS622-MC-MMAE | 10:1 | 5.72 | 48 | 16 |
| SNS622-MC-vc-PAB-DM1 | 10:1 | 6.56 | 123 | 55 |
| SNS622-MC-vc-PAB-MMAE | 10:1 | 6.31 | 77 | 22 |
| SNS622-MC-DM1 | 10:1 | 5.81 | 64 | 36 |

### Example 2

### In Vitro Anti-Cancer Efficacies of Anti-HAAH Drug Conjugates

All of the anti-ASPH ADCs showed anti-cancer activity when tested using an MTS assay with the human large cell lung carcinoma cell line H460 cells or with the adult acute myeloid leukemia cell line MOLM-14.

Human H460 cells were obtained from the American Type Culture Collection (ATCC; Manassas, Va., U.S.A.). The H460 cells were seeded at a density of 5,000 cells/well in 96-well plates and cultured in HG-DMEM (ThermoFisher Scientific, Waltham. Mass., U.S.A.; Catalog No. 11965-092) supplemented with 20 mM HEPES, 4 mM L-glutamine, 1 mM sodium pyruvate, 1× non-essential amino acids, 50 μg/ml gentamicin, and 10% fetal bovine serum (FBS)). Human MOLM-14 cells were obtained from Dr. Ashkan Emadi at University of Maryland (Baltimore. Md., U.S.A.). The MOLM-14 cells were seeded at a density of 5000 cells/well in 96-well plates and cultured in RPMI 1640 with 20% heat-inactivated FBS. The H460 and MOLM-14 cells were treated with naked antibodies or ADCs for 24 hours, starting at the time of cell seeding. By the end of experiment, the cell viability for both cell lines was determined quantitatively using CELL TITER 96 AQUOUS ONE SOLUTION cell proliferation assay, a colorimetric method for determining the number of viable cells (MTS; Promega, Madison. Wis., U.S.A.; Catalog No. G3582).

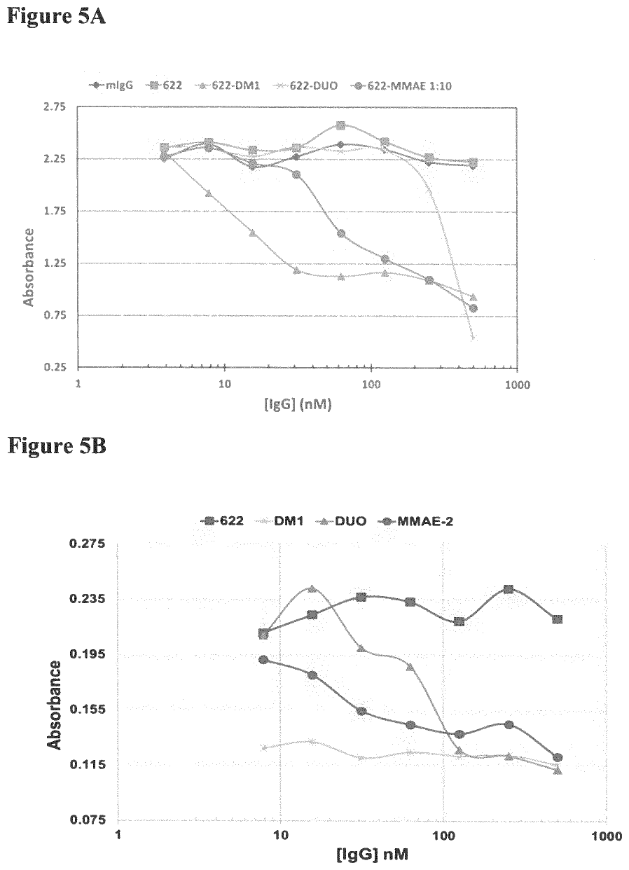

As seen in FIG. 5A, no changes in the viability of the H460 cells was seen when using MTS analysis with either the cancer-specific antibody SNS622, or the non-specific IgG1 antibody. This figure also shows that SNS622-MC-DM1, SNS622-MC-vc-PAB-MMAE, and SNS622-MC-PEG4-vc-PAB-DMEA-DUO all decreased the H460 cell viability. The anti-cancer activity of the antibodies was assessed by the reduction in absorbance at 490 nm, which is a measurement of cell viability using the MTS assay. The highest anti-cancer activity in H460 cells was seen with SNS622-MC-DM1, with an EC50 of ˜15 nM. Showing a slightly lower anti-cancer activity in H460 cells was SNS622-MC-vc-PAB-MMAE, with an EC50 of ˜60 nM, The lowest effect on survival of H460 cells was seen with SNS622-MC-PEG4-vc-PAB-DMEA-DUO, with an EC50 of ˜300 nM.

In this comparison study, neither IgG1 nor SNS622 alone induced growth suppression of H460. Similarly, SNS622 did not suppress the in vitro growth of MOLM-14 either. In contrast, SNS622-drug conjugates SNS622-MC-DM1, SNS622-MC-vc-PAB-MMAE and SNS622-MC-PEG4-vc-PAB-DMEA-DUO all inhibit in vitro cell growth of both H460 and MOLM-14, providing a possibility for treating both HAAH-expressing solid tumors and blood cancers with each of them in vivo. SNS622-MC-DM1 demonstrated the stronger effect in suppressing cell growth of both cancer cell lines (IC₅₀: 15 nM) than SNS622-MC-vc-PAB-MMAE (IC₅₀: 60 nM), although SNS622-MC-DM1 has a less final DAR value than SNS622-MC-vc-PAB-MMAE. SNS622-MC-vc-PAB-MMAE has a 1.8-fold DAR value of that of SNS622-MC-PEG4-vc-PAB-DMEA-DUO, but IC₅₀ of the latter is 5-fold of the former in suppression cell growth of H460. This results indicate that MC vc-PAB-MMAE is more cytotoxic than MC-PEG4-vc-PAB-DMEA-DUO when conjugated to SNS622.

As seen in FIG. 5B, and similar to its activity on H460 cells, there were little to no changes in MOLM-14 cell viability when the cells were treated with SNS622. In comparison with the group of cells treated with SNS622, treating with SNS622-MC-DM1, at a concentration as low as 8 nM, achieved the greatest suppression in the growth of MOLM-14 cells. With an IC₅₀ of ˜50 nM, SNS622-MC-vc-PAB-MMAE showed an effective suppression of MOLM-14 growth. Showing the least effect on the survival of MOLM-14 cells was SNS622-MC-PEG4-vc-PAB-DMEA-DUO, with an IC₅₀ of ˜60 nM. Although all SNS622-conjugated ADCs showed anticancer activities, SNS622-DM1 was chosen for further studies.

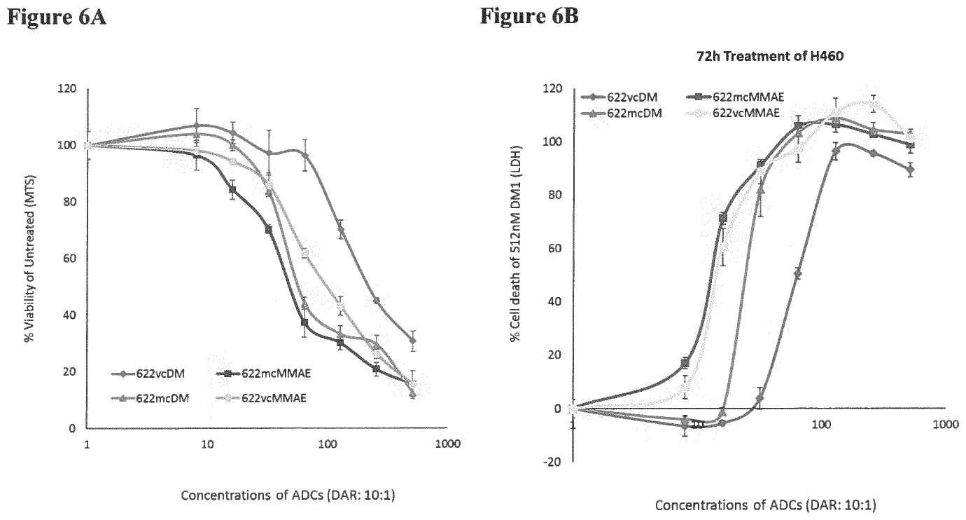

FIG. 6 depicts the effects of linker and payload on cytotoxicity to cancer cell H460. The H460 cells were treated for 72 hours with ADCs (all with reaction DAR at 10:1; See Table I, above, for the final DAR for each ADC), including SNS622-MC-DM1 (▴), SNS622-MC-vc-FAB-DM1 (♦), SNS622-MC-vc-PAB-MMAE (●) and SNS622-MC-MMAE (▪) The cell viability and cell death were separately quantified with MTA (FIG. 6A) and lactate dehydrogenase (LDH) release assays (FIG. 6B), and expressed as percentage of untreated control and percentage of 512 nM DM1-elicited maximal LDH release, respectively. The Log of the antibody concentration in nM is shown on the X axis. The MTS results shown that SNS622 conjugated with DM1 (IC₅₀: 64 nM) or MMAE (IC₅₀: 48 nM) with a linker of MC always showed stronger cytotoxicity than a linker of MC-vc-PAB (IC₅₀ₛ 123 and 77 for payloads DM1 and MMAE, respectively). Similarly, the LDH analysis shown that SNS622 conjugated with DM1 (LC₅₀: 36 nM) or MMAE (LC₅₀: 16 nM) with a linker of MC always showed stronger cytotoxicity than a linker of MC-vc-PAB (LC₅₀ₛ 55 and 22 for payloads DM1 and MMAE, respectively), Furthermore, MMAE as payload showed stronger cytotoxicity than DM1 when each is conjugated to SNS622 with same linker. With MC as a linker, SNS622 conjugated with MMAE resulted in higher cytotoxicity (IC₅₀: 48 nM; LC₅₀: 16) than it conjugates to DM1 (IC₅₀: 64 M; LC₅₀: 36). Similarly, with MC-vc-PAB as a linker, SNS622 conjugated with MMAE again resulted in higher cytotoxicity (IC₅₀: 77 nM; LC₅₀: 0.22) than it conjugates to DM1 (IC₅₀: 123 nM; LC₅₀: 55).

### Example 3

### SNS622-DM1 Drug-Antibody Ratio (DAR) Response

This example shows that the cytotoxic activity of SNS622-DM1 is dependent on the number of conjugated DM1, but there appears to be no correlation with the pH of the ADC. To determine if the cytotoxic activity is dependent on the drug antibody ration (DAR) or of the pH of the ADC, SNS622-MC-DM1s were synthesized by Goodwin Biotechnology using drug antibody ratios (DARs) of 2.5:1; 5:1; and 10:1; at pH 7 or pH8. The bioactivities of these SNS622 ADCs were examined using H460 cells. The cells were treated for 48 hours with these SNS622-MC-DM1s, or with SNS622 as control. Cell viability was evaluated at the end of the experiment using an MTS assay.

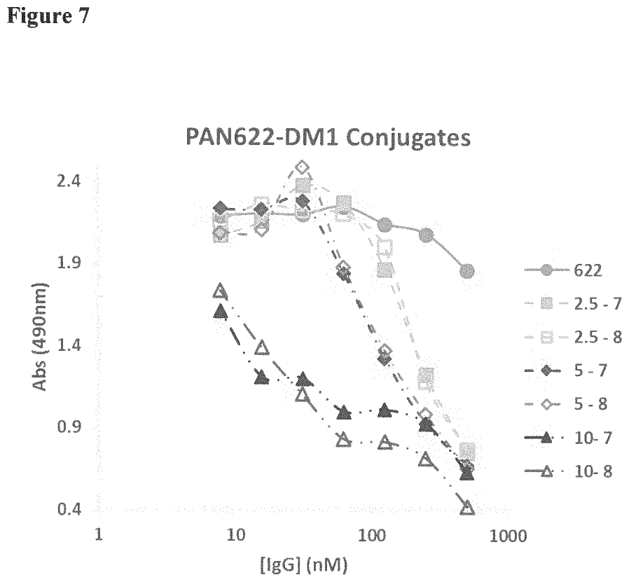

The final DAR for after each conjugation reaction is shown in Table III, below. The effects on H460 cell viability were determined with MTS assay after a 48-hour treatment, and the results are shown in FIG. 7. The absorbance at 490 nm is shown on the Y axis. The Log of the antibody concentration in nM is shown on the X axis. The results demonstrated that cytotoxicity is positively related to DAR, and less related to pH. Table III, below, presents the reaction DAR and pH, and the final DAR for SNs622-DM1 ADCs tested.

TABLE III
DAR and pH at Reaction and BAR at
Final Product for SNS622-MC-DM1

| Name | Reaction DAR | pH | Final DAR |
|---|---|---|---|
| SNS622-DM1A1 | 2.5:1 | 7 | 2.00 |
| SNS622-DM1A2 | 2.5:1 | 8 | 2.10 |
| SNS622-DM1B1 | 5:1 | 7 | 3.22 |
| SNS622-DM1B2 | 5:1 | 8 | 3.36 |
| SNS622-DM1C1 | 10:1 | 7 | 5.08 |
| SNS622-DM1C2 | 10:1 | 8 | 5.81 |

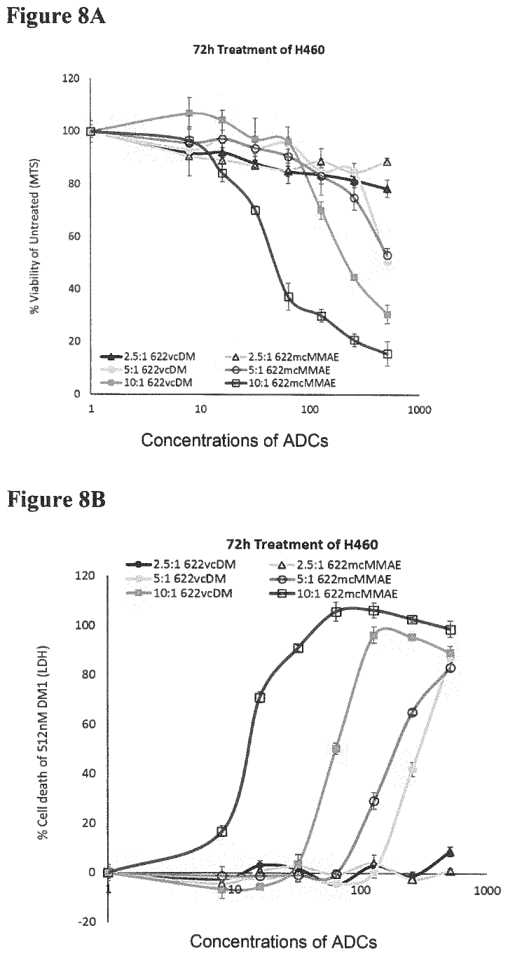

SNS622-MC-vc-PAB-DM1 and SNS622-MC-MMAE were also synthesized under different initial DAR ratios of 2.5:1, 5:1 and 10:1 at fixed pH8. The final DAR for each after conjugation reactions is shown in Table IV, below. The 460 cells were treated for 72 hours with ADCs SNS622-MC-vc-PAB-DM1 at with OAR 2.5:1; 5:1; and 10:1, or SNS622-MC-MMAE at with DAR 2.5:1; 5:1; and 10:1. The cell viability and cell death were separately quantified with MTA and lactate dehydrogenase (LDH) release assays. FIG. 8A depicts a graph of the MTA quantification results expressed as percentage of untreated control. FIG. 8B depicts a graph of the LDH assay results expressed as percentage of 512 nM DM1-elicited maximal LDH release. The Log of the antibody concentration in nM is shown on the X axis. Again, for each of these ADCs, cytotoxicity is positively related to DAR, and the most potent ADCs were those with DAR of 10:1.

TABLE IV
SNS622-MC-MMAE and SNS622-MC-vc-PAB-
DM1 DAR at Reaction and Final Product

| Name | ReactionDAR | Final DAR |
|---|---|---|
| PAN622-MC-MMAE | 2.5:1 | 0.27 |
| PAN622-MC-MMAE | 5:1 | 4.59 |
| PAN622-MC-MMAE | 10:1 | 5.72 |
| PAN622-MC-vc- PAB-DM1 | 2.5:1 | 3.01 |
| PAN622-MC-vc-PAB-DM1 | 5:1 | 4.23 |
| PAN622-MC-vc-PAB-DM1 | 10:1 | 6.56 |

As seen in FIG. 7, regardless of the DAR or pH, all of the SNS622-MC-DM1 possess anti-cancer activity. The potency of the anti-cancer activity depends on the DAR; i.e., the SNS622-MC-DM1 with a 10:1 DAR showed the highest efficacy in the suppression of H460 cell viability, while the SNS622-MC-DM1 with a 2.5:1 DAR showed the lowest efficacy in suppression of H460 cell viability There appeared to be little to no difference between the results obtained for SNS622-MC-DM1 at pH 7 and SNS622-MC-DM1 at pH 8. In view of these results, together with the superior cytotoxicity to MOLM-14 cancer cells, SNS622-MC-DM1 with a 10:1 DAR and pH 8 was chosen for additional studies.

### Example 4

### Specificity of SNS622-MC-DM1 to Cancer Cells

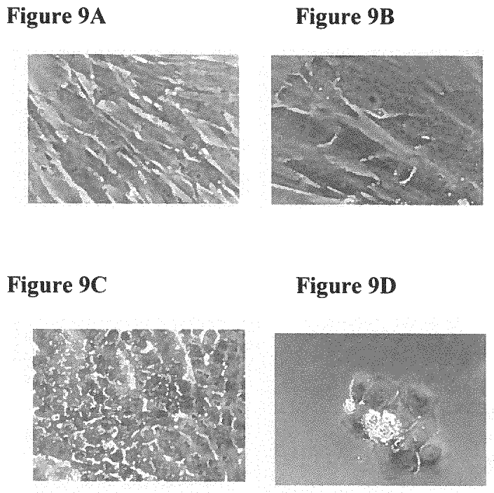

HAAH is highly expressed on the surface of cancer cells, but not on normal adult human cells. The specificity of SNS622-MC-DM1 was examined using human lung carcinoma cell line H460 and LONZA NHLF, a normal human lung fibroblasts (NHLF) cell line. Both cell lines were cultured in HG-DMEM (ThermoFisher Scientific Catalog No. 11965-092) supplemented with 20 mM HEPES (4-(2-hydroxyethyl)-1-piperazineethanesulfonic acid), 4 mM L-glutamine, 1 mM sodium pyruvate, 1× non-essential amino acids, 50 μg/ml gentamicin and 10% fetal bovine serum (FBS). H460 and NHLF cells were seeded into 96-well plates at 5,000 cells per well, following a 2-day FBS deprivation, and released by culture in complete culture medium containing 10% FBS. The cells were allowed to grow for 2 days after releasing, and then treated with or without 32 nM SNS622-MC-DM1. By 72 hours post treatment, cell death was determined by observing cell morphology using a phase contrast microscope at 150×. As seen in FIG. 9D, treatment of H460 cells with 32 nM SNS622-MC-DM1 resulted in reduction of cell density, while no significant reduction of cell density was observed when NHLF cells were treated with the same SNS622-MC-DM1 concentration (FIG. 9B). Morphologically, the dead cells appear as bright shrunk particles (FIG. 9D). FIG. 9A shows an image of untreated NHLF cells, and FIG. 9C shows an image of untreated H460 cells.

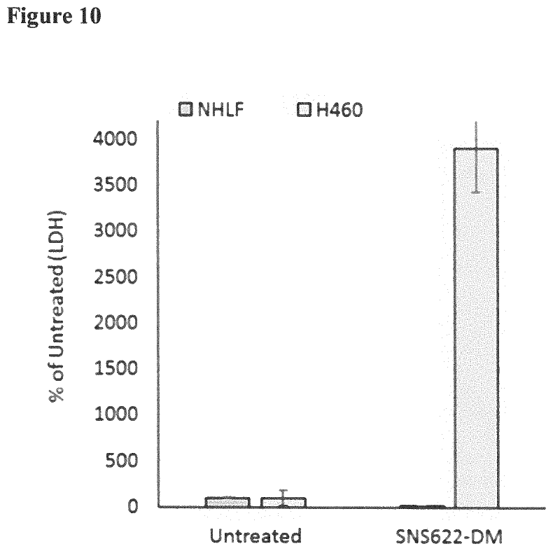

The cell death was also quantified using lactate dehydrogenase (LDH) releasing assay. For the LDH assay, a mixture of a 35 μl aliquot of culture medium and 17.5 μl of substrate, enzyme, and dye solutions (Sigma, St. Louis, Mo., U.S.A.) was incubated at room temperature for 30 minutes, and absorbance was measured at 490 nm using a plate reader (ELx805; BioTek, Winooski, Vt., U.S.A.). As seen on FIG. 10, treatment of H460 cells with 32 nM SNS622-MC-DM1 elicited a 39× increase in release of LDH when compared to untreated H460 cells, indicating leakage of cell membrane. In contrast, treatment of NHLF cells with 32 nM SNS622-MC-DM1 did not result in a detectable increase in LDH release. These results demonstrate a specificity of SNS622-MC-DM1 to cancer cells, laying a basis for its application in cancer specific-targeting therapy.

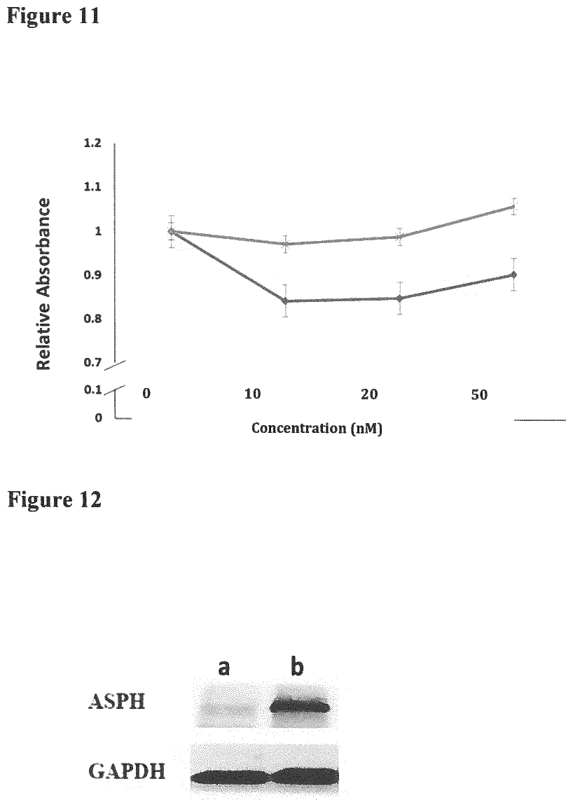

The in vitro specificity of SNS622-MC-DM1 was also examined using the human pancreas cell line MIA-PaCa-2 ASPH. This cell line has a high level expression of ASPH. MIA-PaCa-2 cells were seeded at a density of 2,000 cells/well and cultured in high-glucose (25 mM D-glucose) Dulbecco's Modified Eagle's Medium (DMEM) with 10% FBS at 37° C. in a humidified atmosphere containing 5% CO₂. The cells were treated with 10 nM IgG1-MC-DM1, 20 nM IgG1-MC-DM1, 50 nM IgG1-MC-DM1, 10 nM SNS622-MC-DM1, 20 nM SNS622-MC-DM1, or 50 nM SNS622-MC-DM1 for 48 hours. IgG1-MC-DM1 is a control ADC obtained from Goodwin Biotechnology, having catalog No. DAGIC1194, The cytotoxicity of the ADCs was measured using an MTS assay kit (Promega, Catalog No, G3582). After treatment for 1 hour with the MTS solution, absorbance was measured using a microplate reader at a 490 nm wavelength, with a reference at 690 nm. As seen in FIG. 11, treatment with SNS622-MC-DM1 resulted in a reduction of about 16% relative absorbance, reflecting a significant decrease in MIA PaCa-2 cell viability. In contrast, treatment with IgG1-MC-DM1 at the same concentrations showed no effect on survival of the MIA PaCa2 cells.

### Example 5

### Specificity of SNS622-MC-DM1 In Vivo

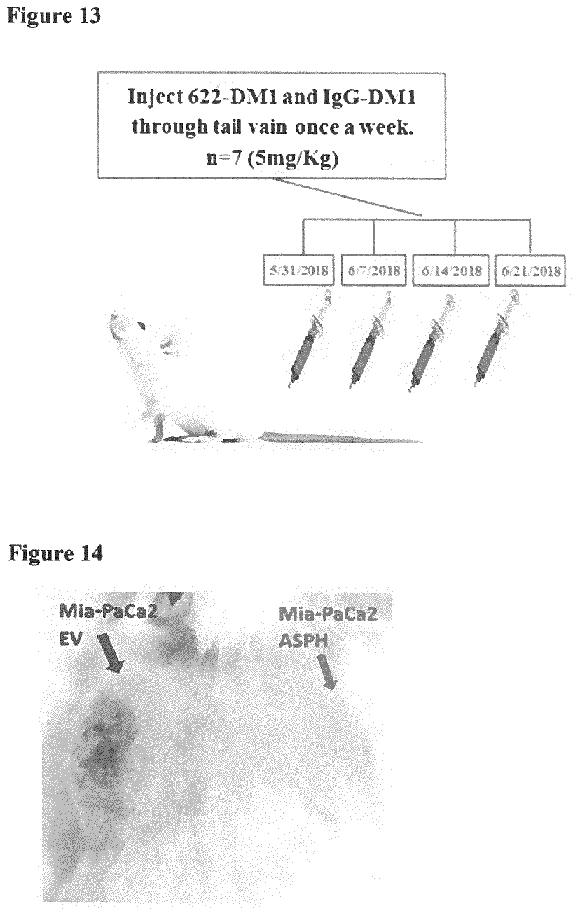

The efficacy and specificity of SNS622-MC-DM1 was first examined in the mouse model with inoculation of pancreas cancer cells. In this model. 5 to 6-week-old female Nod Scid Gamma (NSG) mice (The Jackson Laboratory, Bar Harbor, Me., U.S.A.) were subcutaneously inoculated with 5×10⁵ MIA-PaCa-2 EV cells (MIA-PaCa-2 cells transduced with an empty lentivirus vector) in 100 μl buffer solution on one shoulder, and with 5×10⁵ MIA-PaCa-2 ASPH cells (MIA-PaCa-2 cells expressing high levels of ASPH) in 100 μl buffer solution on the other shoulder Two weeks post-inoculation, SNS622-MC-DM1 or IgG1-MC-DM1 was injected at 5 mg/kg through a tail vein once a week for 4 weeks. A representation of the schedule used for the ADC treatments is show n in FIG. 13.

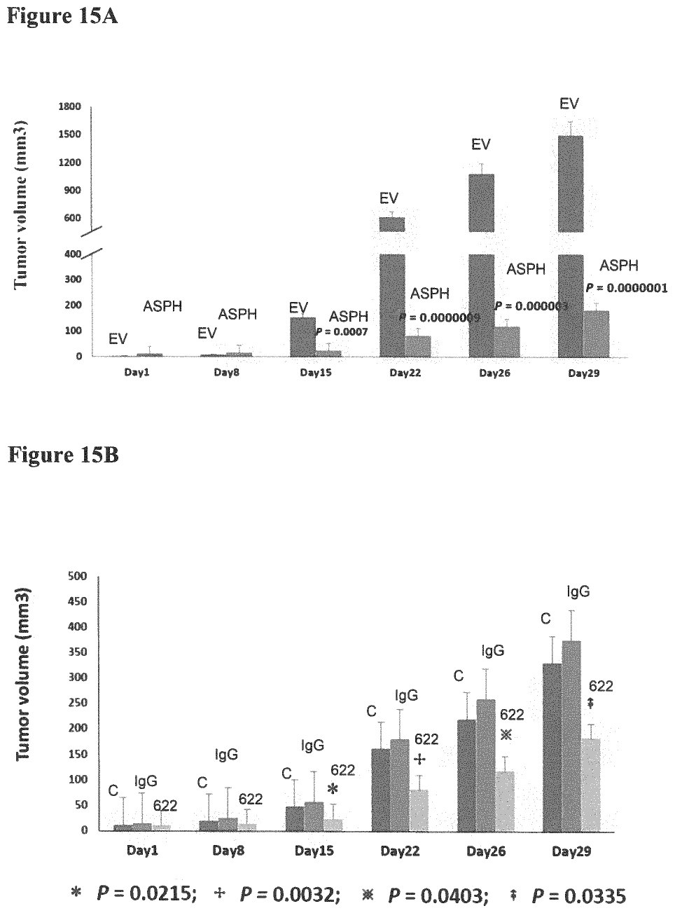

The volume of the tumors inoculated with the MIA-PaCa-2 ASPH cells was measured starting at day 1 of treatment with ADC, and the results are shown in FIG. 15B. There was no reduction in tumor volume in the vehicle-treated control group, or the group treated with the non-specific ADC (IgG-MC-DM1). In contrast, in comparison with the IgG-MC-DM1 treated group, treatment with 5 mg/ml SNS622-MC-DM1 resulted in approximately 55% reduction in tumor volume by day 15; in approximately 53% reduction in tumor volume by day 22; and in about 53% by day 29.

The expression of ASPH in MIA-PaCa-2 ASPH (also referred to here as MIA-PaCa-2 HAAH) cells (MIA-PaCa2 cells transformed to express high levels of HAAH), and MIA-PaCa-2 EVs cells (MIA-PaCa-2 cells transduced with an empty lentiviral vector) was studied using western blot analyses (FIG. 12). The pancreas cancer cell line MIA-PaCa-2 ASPH showed high ASPH expression, while the MIA-PaCa-2 EV showed very low ASPH expression. Glyceraldehyde-3-phosphate dehydrogenase (GAPDH) was used as a loading control.

The response to treatment with SNS622-MC-DM1 of tumors derived from MIA-PaCa-2 HAAH cells and from MIA-PaCa-2 EV cells were compared in the same mouse. FIG. 14 shows images from the inoculated tumors in each side a mouse. These images show that treatment with 5 mg/kg SNS622-MC-DM for 29 days strongly suppressed growth of MIA-PaCa-2 ASPH-derived tumors, but had a much lower effect on tumors derived from MIA-PaCa-2 EV cells.

The tumors were measured, and a graph of their volumes is shown in FIG. 15A. As seen on this figure, the volume of the MEA-PaCa-2 EV-derived tumors grew fast throughout the duration of the experiment, although the mice received SNS622-MC-DM1 treatment. In contrast, the MIA-PaCa-2 ASPH-derived tumors were robustly suppressed with SNS622-MC-DM1 treatment. By treatment day 29, the volume of the MIA-PaCa-2 ASPH-derived tumors was approximately 1/7 the volume of the MIA-PaCa-2 EV-derived tumors.

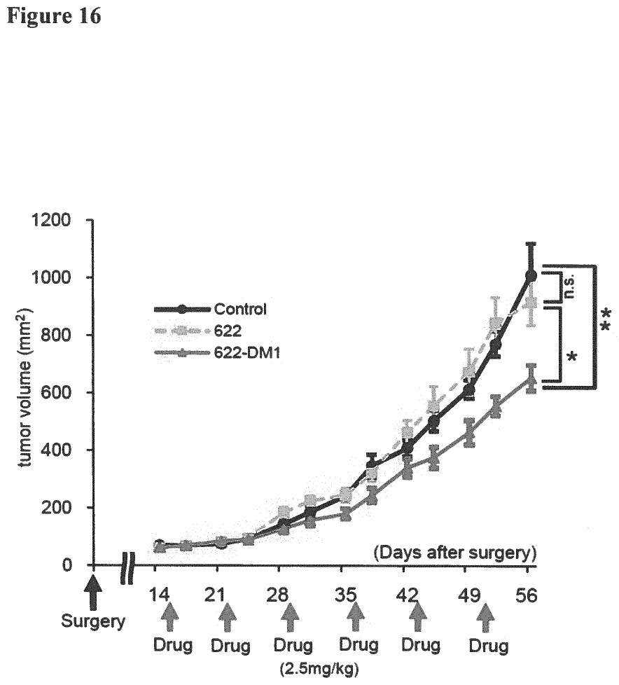

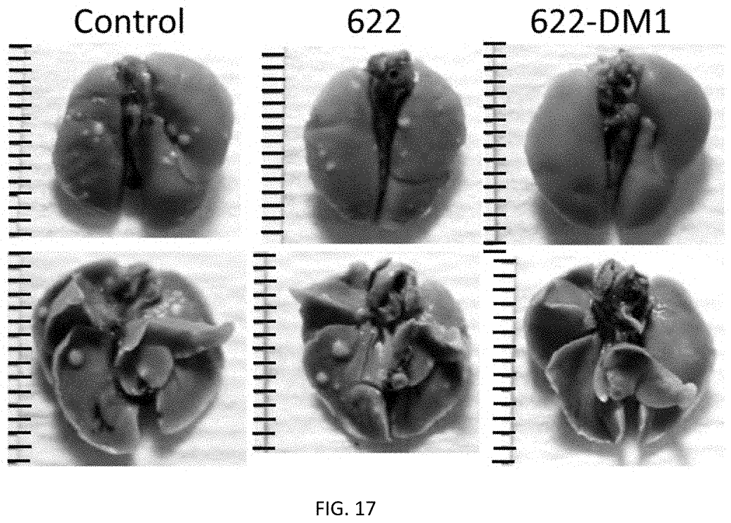

Pancreatic ductular adenocarcinoma (PDAC) is a highly lethal malignancy with limited treatment options. HAAH is a cell surface protein that is highly expressed in 97.1 of PDACs. The specificity of SNS622-MC-DM1 was next examined in the patient-derived xenograft (PDx) mouse model PDAC. The patient-derived xenograft (PDx) murine model was established by using surgically resected PDAC (from Rhode Island Hospital, Providence, R.I., U.S.A.). Five to six-week-old female Nod Scid Gamma (NSG) mice were employed. The surgically resected tumor tissue was diced into 5×5×5 mm³ pieces, and a piece of tumor tissue was subcutaneously transplanted under anesthesia with isoflurane into each mouse via a small incision in the lower back, A buprenorphine analgesic was injected to each mouse for 3 days after surgery. The SNS622 antibody or the SNS622-MC-DM1 ADC was administered at a dose of 2.5 mg/kg by intravenous tail-vein injection every two weeks starting at day 17 post-surgery. Body weight and tumor size were measured twice a week and tumor volume was calculated using the modified ellipsoid formula (0.5×length×width²). To determine the effect of SNS622-MC-DM1 on lung metastasis, all lung tissues were immersed in Bouin's fixative solution (Sigma-Aldrich, St. Louis, Mo., U.S.A.; catalog No, HT10132) and metastatic nodules on the surface of the lungs were counted. Animal experiments were conducted in accordance with the guidelines of the Institutional Animal Care and Use Committee (IACUC) at Rhode. Island Hospital. As seen on FIG. 16, treatment of mice with SNS622-MC-DM1 significantly reduced PDAC tumor growth by day 35 compared to the PDAC tumor growth on mice treated with the SNS622 antibody or the non-treated control.

### Example 6

### Inhibition of Lung Metastasis in a PDx Model of PDAC by SNS622-DM1

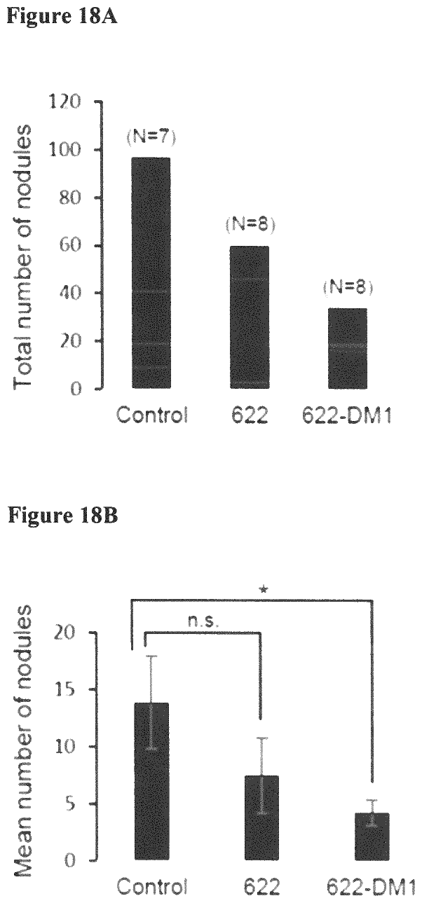

The PDx model was established from an individual with PDAC that exhibited spontaneous lung metastasis. This phenotype was maintained in the PDx model of NSG mouse. Serial passage of this PDAC in NSG mice confirmed the durability and transmissibility of this metastatic phenotype from the F2 to the F9 generation. Therefore, to evaluate the effect of SNS622-MC-DM1 administration on metastatic spread to the lung, this PDx model of PDAC w as employed. In this context, dose and period of administration of PAN-622 or SNS622-MC-DM1 for treatment of NSG mice was reduced to 1.9 mg/kg and the development and growth of metastatic nodules in the lungs after the treatment was assessed After 6 weeks of administration, the number of macroscopic metastatic nodules on the lung surface was determined. As seen in FIG. 18A, the total number of nodules in control mice was close to 100, the total number of nodules in mice treated with SNS622 was around 60, and the total number of nodules in mice treated with SNS622-MC-DM1 was approximately 35. As seen in FIG. 18B, the mean number of nodules was 13.9 per animal in the control untreated group, and the mean number of nodules was 3.25 per animal in the group treated with SNS622-MC-DM1 ADC (P=0.024).

### Example 7

### Binding of SNS622-DM1 to H460 Lysate and to the HAAH Catalytic Domain

To describe the specificity of SNS622-DM1 further, a binding assay of naked antibodies and ADCs against H460 lysate was conducted.

H460 cells were harvested, the cell density was adjusted to 2×10⁶ cells/ml with PBS, and the resuspended cells sonicated on ice twice in 30 second pulses. After centrifugation at 300×g for 5 minutes the supernatant was collected and the protein concentration measured using a NANODROP spectrophotometer (ThermoFisher Scientific). A 96-well plate was coated with H460 lysate and dried at room temperature overnight. Following a 1-hour blockage with 1% non-fat dry milk in phosphate buffered saline (PBS), the plates were incubated for 45 minutes at 37° C. with each either 0.5 μg/ml IgG1λ (non-specific monoclonal antibody), IgG1λ-MC-DM1 (non-specific ADC), or SNS622-MC-DM1, or with 5 μg/ml SNS622. Following five PBS rinses to remove un-bound antibody. 1:100 goat anti-human IgG (8)-HRP was added and the plates incubated for 30 minutes at 37° C. Following five PBS rinses TMB substrate was added and, after incubation of the plates for 15 minutes at room temperature. Stop solution was added to terminate the reactions. The plates were read at 450 nm using a 96-well plate reader (ELx808, BioTek).

Table V, showing the percent of loading amount bound is shown below. Table V shows that SNS622 and SNS622-MC-DM1 demonstrated a high binding specificity to the H460 lysate (92% and 107%, respectively). In contrast, binding of IgG1λ and IgG1λ-MC-DM1 to the H460 lysate was minimal (<0.002% for both).

TABLE V
Binding of Naked Antibodies and ADCs to H460 Cells

| Sonicated H460 | | | | | |
|---|---|---|---|---|---|
| (2E⁶ cells/ml) | SNS622-DM1 | | IgG1λ-DM1 | SNS622 | IgG1λ |
| Loading amount | 5.0 | μg/ml | 5.0 μg/ml | 0.5 | 5.0 |
| Binding amount | 5.35 | μg/ml | <0.001 | 0.46 | <0.001 |

| | | | | |
|---|---|---|---|---|
| Percent binding | 107 | <0.002 | 92 | <0.002 |

The catalytic domain of HAAH (HAAH-cat) is exposed at the surface of HAAH-expressing cancer cells. The binding of SNS622-MC-DM1 at different drug-antibody ratio (DAR) and pH to HAAH-cat was measured using IgG1λ as control. The plates were coated with HAAH by treating with 4 λg/ml HAAH catalytic domain overnight. Following blocking with 1% Non-Fat Dry Milk in PBS for one hour, the plates were incubated for 45 minutes at 37° C. with 11.1 mg/ml IgG1λ; 9.6 mg/ml SNS622; 4.8 mg/ml SNS622-MC-DM1 at DAR 2.5, pH 7.0; 5.9 mg/ml SNS622-MC-DM1 at DAR 2.5, pH8.0; 6.1 mg/ml SNS622-DM1 at DAR 5.0, pH 7.0; 5.7 mg/ml SNS622-MC-DM1 at DAR 5.0, pH8.0; 4.1 mg/ml SNS622-DM1 at DAR 10.0, pH7.0; or 4.4 mg/ml SNS622-MC-DM1 at DAR 10.0, pH8.0. Following 5 PBS rinses the plates were incubated with 1:100 goat anti-human IgG (8)-HRP for 30 minutes at 37° C., Following five PBS rinses TMB substrate was added, and after a 15-minute incubation at room temperature, Stop solution was added to terminate the reactions. The plates were read at 450 nm using a 96-well plate reader (ELx808, BioTek). The binding results are shown below in Table VI

TABLE VI
Binding of SNS622-MC-DM1 at Different DAR and pH to HAAH-cat

| Sample | SNS622-MC-DM1 Conjugate | | | | | | SNS622 | |
|---|---|---|---|---|---|---|---|---|
| DM1: Ab | 2.5 | 2.5 | 5.0 | 5.0 | 10.0 | 10.0 | Standard | IgG1λ |
| pH | 7.0 | 8.0 | 7.0 | 8.0 | 7.0 | 8.0 | | |
| [Protein] | 4.847 | 5.876 | 6.059 | 5.704 | 4.095 | 4.438 | 9.55 | 11.1 |
| (mg/ml) | | | | | | | | |
| [Average]* | 3.64 | 4.98 | 4.92 | 5.11 | 4.66 | 3.57 | 9.32 | <0.001 |
| (mg/ml) | | | | | | | | |
| % Binding | 75.12 | 84.81 | 81.15 | 89.52 | 11.69 | 80.47 | 97.60 | 0.01 |

The results show that almost 98% of SNS622 binds to the HAAH catalytic domain (HAAH-cat), while less than 1% of the non-specific antibody IgG1λ binds to the catalytic domain (<0.01%), Binding by SNS622-MC-DM1 s at different DARs and pH still keep high affinity to the catalytic domain (75-114%).

In general, these results in binding assays demonstrate high specific binding of SNS622-DM1 to the HAAH-expressing cancer cell line H460 lysate and to HAAH-cat. In contrast, nonspecific IgG1λ and IgG1-MC-DM1 have minimal binding to H460 lysate and to HAAH-cat. These provide the underlying mechanism for the in vitro and in vivo specificity of SNS622-MC-DM1 against cancer but not normal cells.

While the invention has been described with reference to certain exemplary embodiments thereof, those skilled in the art may make various modifications to the described embodiments of the invention without departing from the scope of the invention. The terms and descriptions used herein are set forth by way of illustration only and not meant as limitations. In particular, although the present invention has been described by way of examples, a variety of compositions and processes would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

What is claimed is:

1. An antibody-drug conjugate comprising an anti-HAAH monoclonal antibody conjugated to at least one cytotoxic or cytostatic drug, and optionally a linker connecting the antibody and the cytotoxic or cytostatic drug.

2. The antibody-drug conjugate of claim 1, wherein the anti-HAAH antibody is SNS622.

3. The antibody-drug conjugate of claim 1, wherein the at least one drug is selected from an auristatin derivative, a maytansinoid and/or a duocarmycin derivative DUO.

4. The antibody-drug conjugate of claim 1, wherein the auristatin derivative is selected from MMAE or MMAF.

5. The antibody-drug conjugate of claim 1, wherein the maytansinoid is selected from DM1 or DM4.

6. The antibody-drug conjugate of claim 1, wherein the duocarmycin derivative is DUO.

7. The antibody-drug conjugate of claim 1, comprising at least one linker connecting the antibody to the drug.

8. The antibody-drug conjugate of claim 7, wherein the at least one linker is selected from the group consisting of maleimidocaproyl (MC), valine citruline (vc), para amino benzyl alcohol (PABA), a 4-unit polyethylene glycol (PEG4), and N,N-Dimethylethanolamine (DMEA).

9. A method of making an antibody-drug conjugate comprising, conjugating at least one cytotoxic or cytostatic drug to an anti-HAAH antibody, and optionally conjugating a linker connecting the drug and the antibody.

10. The method of claim 9, wherein the anti-HAAH antibody is SNS622.

11. The method of claim 9, wherein the at least one cytotoxic or cytostatic drug is an auristatin derivative.

12. The method of claim 9, wherein the at least one cytotoxic cytostatic drug is a maytansinoid

13. The method of claim 9, comprising at least one linker connecting the antibody to the drug.

14. The method of claim 13, wherein the at least one linker is selected from the group consisting of maleimidocaproyl (MC), valine citruline (vc), para-amino benzyl alcohol (PABA), a 4-unit polyethylene glycol (PEG4), and N,N-Dimethylethanolamine (DMEA).

15. The method of claim 14, wherein the at least one linker is valine citruline-para-amino benzyl alcohol (vc-PAB).

16. The method of claim 14, wherein the linker is maleimidocaproyl (MC).

17. A pharmaceutical composition comprising the antibody-drug conjugate of claim 1 and a pharmaceutically acceptable medium.

18. A method of treating a patient in need thereof, comprising administering to the patient the pharmaceutical composition of claim 17.

19. A method of treating cancer, comprising contacting a cancer with the antibody drug conjugate of claim 1.

20. The method of claim 19 in which the cancer is selected form pancreatic, lung, breast or AML.


SEQUENCE LISTING (5 sequences)

SEQ ID NO: 1 - protein, 455 aa, Artificial Sequence
  Heavy Chain
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARTGYSSSWVVNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 216 aa, Artificial Sequence
  Light Chain
QPVLTQSPSASGTPGQRVTISCSGSSSNIGSNYVYWYQQLPGTAPKLLIYKNNQRPSGVPDRFSGSKSGTAASLAISGLQSEDEADYYCAAWDDSLRGYVFGTGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 15 aa, Artificial Sequence
  Light chain CDR1
SGSSSNIGSNYVYWY

SEQ ID NO: 4 - protein, 12 aa, Artificial Sequence
  Light Chain CDR2
KLLIYKNNQRPS

SEQ ID NO: 5 - protein, 11 aa, Artificial Sequence
  Light Chain CDR3
AAWDDSLRGYV